(19) United States
(12) Patent Application Publication
(10) Pub. No.: US 2024/0047079 A1
(43) Pub. Date: Feb. 8, 2024

(54) SYSTEM FOR ESTIMATING FOR INFECTIOUS DISEASE TRANSMISSION

(71) Applicant: FOUNDATION OF SOONGSIL UNIVERSITY-INDUSTRY COOPERATION, Seoul (KR)

(72) Inventors: Minhae KWON, Seoul (KR); Sujin AHN, Seoul (KR)

(73) Assignee: FOUNDATION OF SOONGSIL UNIVERSITY-INDUSTRY COOPERATION, Seoul (KR)

(21) Appl. No.: 17/960,849

(22) Filed: Oct. 6, 2022

(30) Foreign Application Priority Data
Aug. 2, 2022 (KR) 10-2022-0095986

(51) Int. Cl. G16H 50/80

(52) U.S. Cl. CPC G16H 50/80

(57) ABSTRACT

A system for estimating for infectious disease transmission includes: a parameter receiving unit that constructs a discrete-time Markov chain model indicating a state and a state transition probability, and receives a parameter indicating status information according to the infectious disease transmission at a time point after t days have elapsed from a start of infection spread; a calculation unit that calculates the number of hidden infectious states through backward reasoning, and calculates the number of hidden infectious states through forward reasoning using the received parameter; an extraction unit that extracts an inverse scale coefficient using the calculated number of infection states and calculates a reproduction factor using the extracted inverse scale coefficient; and a prediction unit that updates infectious disease status information using the calculated reproduction factor, and predicts the number of hidden infectious persons using the updated infectious disease status information.

### CROSS-REFERENCE TO RELATED APPLICATION

This application is based on and claims priority under 35 U.S.C. §119 to Korean Patent Application No. 10-2022-0095986, filed on Aug. 2, 2022, in the Korean Intellectual Property Office, the disclosure of which is incorporated by reference herein in its entirety.

### BACKGROUND

### 1. Field

One or more embodiments relate to a system for estimating for infectious disease transmission, and more particularly, to a system for estimating for infectious disease transmission which provides a data-based infectious disease model using a Markov chain and uses mathematical modeling partitioned into a plurality of states including a vaccinated state, a hospitalized state, and the like to estimate a risk measurement index and the number of hidden infectious persons for a new infectious disease.

### 2. Description of the Related Art

The infectious disease is a disease caused by infection of a human body with bacteria, virus, fungus, parasite, and the like called a pathogen. Among them, virus species capable of rapid mutation and self-replication have very favorable conditions to cause a global infectious disease pandemic.

The infectious disease, including Ebola, MERS, Zika virus, or the like which is a kind of indigenous infectious disease that occurred in the past, and COVID-19 (corona 19), which is causing a recent pandemic, is rapidly spreading around the world. COVID-19 has spread rapidly since a first confirmed case occurs in 200 seconds, thereby affecting not only the daily life of the people but also the economy and society of the country as a whole.

Therefore, in recent years, in major advanced countries, research is being conducted on infectious disease transmission prediction technology according to a current status and a future prospect of the prediction technology based on big data, high-performance computing, and artificial intelligence.

The development of the prediction technology for infectious disease may be explained by dividing it into three stages. The first is a mathematical algorithm-oriented technology, the second is a prediction technology centered on high-performance computing infrastructure such as supercomputers, and the third is a simulation-based technology through connection with big data that may explain a floating population pattern of an area which is a target of the prediction.

Among them, the reason for mathematically approaching the infectious disease model is as follows.

First, because experimental verification in reality is impossible due to ethical issues, it is necessary to have a process of understanding an infection pattern of the infectious disease by mathematically reproducing the infectious disease model. Second, the mathematical modeling may contribute to predict future numbers in a situation where high-quality empirical data is not enough or the future situation is not predicted due to the sudden outbreak of a new infectious disease, or the situation is high in uncertainty.

Therefore, mathematical modeling of the infectious disease is useful in that it may understand the trends of various diseases and build a system for predicting an epidemiology of the infectious disease.

The mathematical model that may explain the infectious disease transmission process includes compartmental models, and representative compartmental models include Susceptible-Infectious-Recovered (SIR) and Susceptible-Exposed-Infectious-Recovered (SEIR) models.

The compartmental model illustrates that the number of populations in each state changes according to a constant flow. However, the conventional compartmental model is difficult to apply in complex phenomena because it considers only a susceptible person (S), an infectious person (I), and a recovered person (R). For example, according to the current COVID-19 situation, there was a problem that it was difficult to find information about people who were hospitalized and people who received vaccines in the existing infectious disease model.

In addition, the conventional compartmental model determines that an infection transmission rate is always constant and expresses it as a constant. However, COVID-19 is transmitted by air or droplets, and the infection transmission rate varies depending on an amount of contact with a confirmed person, so it has been affected by a distancing policy. Therefore, there was a problem that the infection transmission rate set as the constant could not be applied to COVID-19.

In addition, since the conventional SIR model only provides data on the confirmed person and does not provide information on the person in a exposed state, there is a problem in that it is impossible to observe the number of the population in a latent state.

The technology that is the background of the present disclosure is disclosed in Korean Patent No. 10-2019-0024549 (published on Mar. 8, 2019).

### SUMMARY

As described above, the present disclosure provides a system for estimating for infectious disease transmission which provides a data-based infectious disease model using a Markov chain and uses mathematical modeling partitioned into a plurality of states including a vaccinated state, a hospitalized state, and the like to estimate a risk measurement index and the number of hidden infectious persons for a new infectious disease.

According to an embodiment of the present disclosure for solving the technical problem, there is provided a system for estimating for infectious disease transmission including: a parameter receiving unit that constructs a discrete-time Markov chain model indicating a state and a state transition probability, and receives a parameter indicating status information according to the infectious disease transmission at a time point after t days have elapsed from a start of infection spread; a calculation unit that calculates the number of hidden infectious states through backward reasoning, and calculates the number of hidden infectious states through forward reasoning using the received parameter; an extraction unit that extracts an inverse scale coefficient using the calculated number of infection states and calculates a reproduction factor using the extracted inverse scale coefficient; and a prediction unit that updates infectious disease status information using the calculated reproduction factor, and predicts the number of hidden infectious persons using the updated infectious disease status information.

The parameter may include at least one of the number of daily confirmed persons, the number of daily primary vaccine inoculated persons, the number of daily hospitalized and recovered persons, the number of hospitalized persons, the number of deaths, the number of susceptible persons corresponding to day 0, and the number of exposed persons, the number of recovered persons, the number of vaccinated states, and the number of hidden infectious states.

The calculation unit may calculate the number of hidden infectious states (î_{b}) through the backward reasoning using Equation below:

$${\hat{i}}_{b} ( t ) = \frac{1}{{φ}^{*}} {X}_{IH} ( t ) + {\sum }_{q = 1}^{t - 1} {( ( \frac{1}{{φ}^{*}} - 1 ) ( 1 - γ ) )}^{q + 1} {X}_{IH} ( t - q )$$

here, φ* indicates an arbitrarily assumed detected case ratio and X_{IH} indicates the number of daily confirmed persons.

The calculation unit calculates the number of hidden infectious states (î_{f}) through the forward reasoning using Equation below:)

î_{f} (t; z)=ϵ(1−ϵ)e(t−2)+ϵU(t−2; z)i(t−2)+(1− [symbol] )i(t−1)−(1− [symbol] ) X_{IH} (t−1)

here,

$$\frac{1}{γ}$$

indicates the average infection period, z indicates the inverse scale coefficient,

$$\frac{1}{ϵ}$$

indicates the average latent period, e indicates the exposed person, and i indicates the hidden infectious person.

The extraction unit may calculate a loss function by substituting the number of hidden infectious states (î_{b}) calculated through the backward reasoning and the number of hidden infectious states (î_{f})calculated through the forward reasoning into Equation below:

$$ℒ ( t ; z ) = \{ \begin{matrix}{( {\hat{i}}_{b} ( t ) - {\hat{i}}_{f} ( t ; z ) )}^{2} , & if {\hat{i}}_{f} ( t ; z ) \geq {X}_{IH} ( t ) \\ λ , & if {\hat{i}}_{f} ( t ; z ) < {X}_{IH} ( t )\end{matrix}$$

The extraction unit may extract the inverse scale coefficient (z) by substituting the calculated loss function into Equation below:

$${z}^{*} = arg {min}_{z} ( \frac{1}{T + 1} {\sum }_{t = 2}^{T + 2} ℒ ( t ; z ) )$$

The extraction unit may calculate the reproduction factor (U(t;z)) using Equation below:

$$𝒰 ( t ; z ) = \frac{1}{z} \frac{{X}_{IH} ( t )}{\text{?} ( {X}_{IH} ( t - l ) \text{?} )} \frac{\text{?} - {X}_{SV} ( t )}{\text{?} ( t )} ,$$

$$\text{?} \text{indicates text missing or illegible when filed}$$

here, s indicates the number of susceptible persons, X_{SV} indicates the number of daily vaccine inoculated persons, and N*(t) indicates the total population at the time point t.

The risk estimation unit may acquire each of reproduction factors calculated during a period τ and calculate the average value of the reproduction factors, and determine that the risk for the infectious disease has increased when the calculated average value is greater than the reproduction factor calculated at the time point t.

The prediction unit may use the reproduction factor to update each state data for the vaccinated state, the susceptible state, the exposed state, the hidden infectious state, the hospitalized state, the recovery state, and the dead state, and predict the number of hidden infectious persons at a time point t+1 using the updated state data.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of an infectious disease transmission estimation system according to an embodiment of the present disclosure;

FIG. 2 is a flowchart for explaining a method for estimating infectious disease transmission using an infectious disease transmission estimation system according to the embodiment of the present disclosure;

FIG. 3 is a diagram for explaining a discrete-time Markov chain model constructed in step S210 illustrated in FIG. 2; and

FIG. 4 is a flowchart for explaining a method for evaluating a risk of an infectious disease using an infectious disease transmission estimation system according to the embodiment of the present disclosure.

### DETAILED DESCRIPTION

Hereinafter, preferred embodiments according to the present disclosure will be described in detail with reference to the accompanying drawings. In this process, thicknesses of the lines or sizes of the components illustrated in the drawings may be exaggerated for clarity and convenience of explanation.

In addition, the terms to be described below are terms defined in consideration of functions in the present disclosure, which may vary according to intentions or customs of users and operators. Therefore, definitions of these terms should be made based on the content throughout this specification.

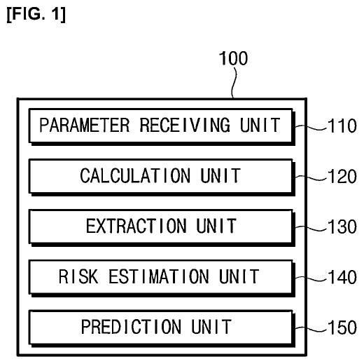

Hereinafter, an infectious disease transmission estimation system according to an embodiment of the present disclosure will be described in more detail with reference to FIG. 1.

FIG. 1 is a configuration diagram of the infectious disease transmission estimation system according to the embodiment of the present disclosure.

As illustrated in FIG. 1, an infectious disease transmission estimation system 100 according to the embodiment of the present disclosure includes a parameter receiving unit 110, a calculation unit 120, an extraction unit 130, a risk estimation unit 140, and a prediction unit 150.

First, the parameter receiving unit 110 receives a parameter indicating status information according to infectious disease transmission at a time point after t days have elapsed from a day when the spread of infection transmission starts, from a server of the Statistics Office in Korea.

The calculation unit 120 calculates the number of hidden infectious states by applying forward reasoning and backward reasoning to the received parameter. In other words, the operation unit 120 calculates the number of hidden infectious states by applying a forward reasoning method that switches in the order of a susceptible person state, a exposed person state, and a hidden infectious state. In addition, the calculation unit 120 calculates the number of hidden infectious states by applying the backward reasoning to the number of hospitalized states.

Next, the extraction unit 130 calculates a loss function using the number of hidden infectious states calculated through the forward reasoning and the number of hidden infectious states calculated through the backward reasoning, and extracts an inverse scale coefficient through the calculated loss function. The extraction unit 130 calculates a reproduction factor by using the extracted inverse scale coefficient.

The risk estimation unit 140 calculates an average value of each calculated reproduction factor for a preset period, and compares the calculated average value with the reproduction factor calculated at the time point t. In comparison, if it is determined that the average value is greater than the reproduction factor, the risk estimation unit 140 determines that the risk for the infectious disease has increased. That is, the risk estimation unit 140 uses the calculated reproduction factor to evaluate the risk for the infectious disease, and outputs the evaluation result.

Finally, the prediction unit 150 uses the reproduction factor calculated at the time point t to update each state data for the vaccinated state, the susceptible state, the exposed state, the hidden infectious state, the hospitalized state, the recovery state, and the dead state. Then, the prediction unit 150 predicts the number of hidden infectious persons at a time point t+1 using the updated state data.

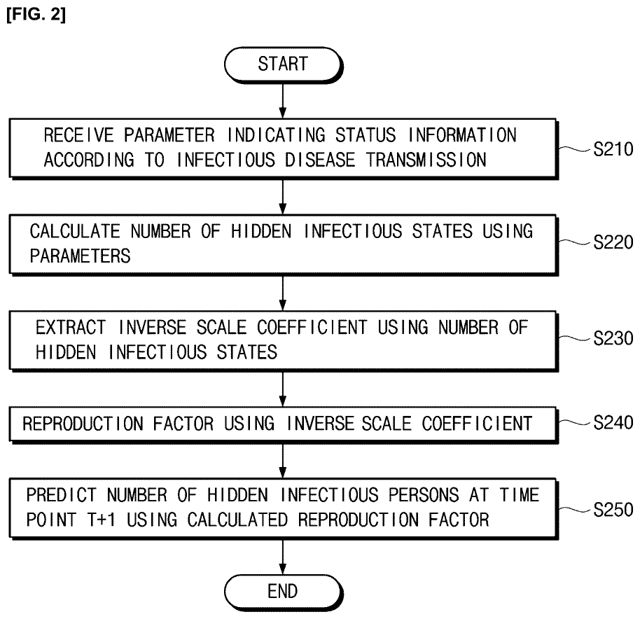

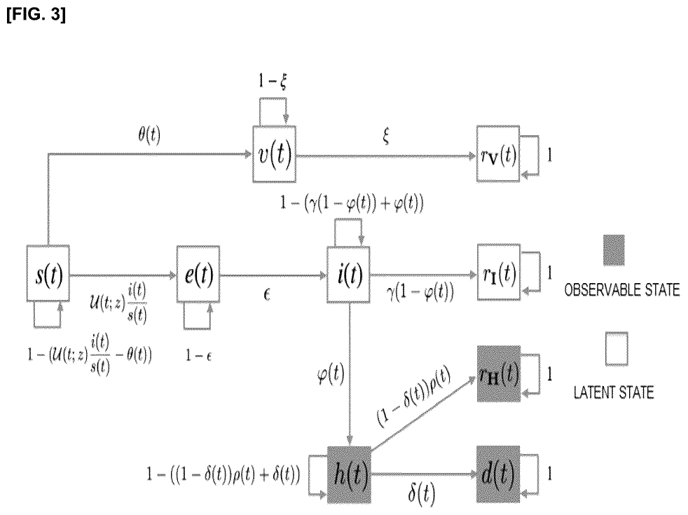

Hereinafter, a method for estimating the infectious disease transmission using the infectious disease transmission estimation system 100 according to the embodiment of the present disclosure will be described in more detail with reference to FIGS. 2 and 3.

FIG. 2 is a flowchart for explaining the method for estimating the infectious disease transmission using the infectious disease transmission estimation system according to the embodiment of the present disclosure.

As illustrated in FIG. 2, the infectious disease transmission estimation system 100 according to the embodiment of the present disclosure receives a parameter indicating status information according to the infectious disease transmission from the Infectious Disease Statistical Office in Korea (S210).

In other words, the parameter receiving unit 110 constructs a discrete-time Markov chain model indicating states and state transition probabilities.

FIG. 3 is a diagram for explaining the discrete-time Markov chain model constructed in step S210 illustrated in FIG. 2.

As illustrated in FIG. 3, the discrete-time Markov chain model indicates states and state transition probabilities. In other words, s(t) indicates the susceptible person state with a probability of being infected in the future at the time point t. At this time, the susceptible person is in a state that has not been infected with the infectious disease or received a vaccine. The susceptible person state (s(t)) may be converted to a v(t) state depending on vaccinated, or may be converted to a exposed state (e(t)) in which it is infected but cannot infect others because it cannot be exposed to the virus.

The exposed state (e(t)) is converted to a hidden infectious state (i(t)) that is not isolated but may transmit the virus to others. In addition, the hidden infectious state (i(t)) may be converted to a state with immunity, that is, a recovered state (ri(t)), or may be confirmed and converted to an hospitalized state (h(t)).

Finally, the hospitalized state (h(t)) may be converted to either a recovered state (r_{H}(t)) or a dead state (d(t)) due to the infection.

When the construction of the discrete-time Markov chain model is completed as described above, the parameter receiving unit 110 receives a parameter indicating status information according to the infectious disease transmission at a time point after t days have elapsed from the start of the infection spread so as to substitute into the discrete-time Markov chain model. Here, the parameter includes at least one of the number of daily confirmed persons, the number of daily primary vaccine inoculated persons, the number of daily hospitalized and recovered persons, the number of hospitalized persons, the number of deaths, the number of susceptible persons corresponding to day 0 (s(0)), and the number of exposed persons (e(0)), the number of recovered persons (r_l(0)), the number of vaccinated states (v(0)), and the number of hidden infectious states (i_b(0)).

When step S210 is completed, the calculation unit 120 calculates the number of hidden infectious states (i(t)) using the input parameters (S220).

In other words, the calculation unit 120 calculates the number of hidden infectious states according to the forward reasoning (î_{f} (t;z) using Equation 1 below.

î_{f}(t; z)=ϵ(1−ϵ)e(t−2)+ϵU(t−2; z)i(t−2)+(1− [symbol] )i(t−1)−(1− [symbol] )X_{IH}(t−1)

Here,

$$\frac{1}{γ}$$

indicates the average infection period, z indicates the inverse scale coefficient,

$$\frac{1}{ϵ}$$

indicates the average latent period, e indicates the exposed person, and i indicates the hidden infectious person. In addition, X_{IH} indicates the number of daily confirmed persons.

For example, it is assumed that t indicates May 5, and t is time point t+2. Then, the number of hidden infectious states (î_{f}(t;z) is calculated using the number of exposed states on May 3 (e(T)), the number of hidden infectious states on May 3 (i(T)), the number of hidden infectious states on May 4 (i(T+1)), and the number of confirmed persons on May 4 (X_{IH}(T+1)).

Then, the calculation unit 120 calculates the number of hidden infectious states (î_{b} (t)) according to the backward reasoning using Equation 2 below.

$${\hat{i}}_{b} ( t ) = \frac{1}{{φ}^{*}} {X}_{IH} ( t ) + {\sum }_{q = 1}^{t - 1} {( ( \frac{1}{{φ}^{*}} - 1 ) ( 1 - ) )}^{q + 1} {X}_{IH} ( t - q ) \qquad [ Equation 2 ]$$

Here, φ* indicates an arbitrarily assumed detected case ratio.

That is, since

$$\frac{1}{{φ}^{*}}$$

plays a role of estimating the total number of infected persons before receiving a confirmed diagnosis,

$$\frac{1}{{φ}^{*}} {X}_{IH} ( t )$$

indicates the total number of infectious persons at the time point t. Therefore î_{b}(t) indicates the sum of the total number of infectious persons at the time point t including the number of confirmed persons and unconfirmed infectious persons, and the number of remaining infectious states at the time point t estimated based on the time before t.

For example, it is assumed that t indicates May 5, and t is time point t+2. Then, the number of hidden infectious states (î_{b}(t)) is calculated using the number of confirmed persons on May 5 (X_{IH}(T+2)) and confirmed person data from the number of confirmed persons on May 2 (X_{IH}(1)) to the number of confirmed persons on May 4 (X_{IH}(T+1)).

When step S220 is completed, the extraction unit 130 extracts the inverse scale coefficient z using the number of hidden infectious states (S230).

First, the extraction unit 130 calculates a loss function ( [symbol] (t;z)) as illustrated in Equation 3 below.

$$ℒ ( t ; z ) = \{ \begin{matrix}{{\hat{i}}_{b} ( t ) - {\hat{i}}_{f} ( t ; z ) )}^{2} , & if {\hat{i}}_{f} ( t ; z ) \geq {X}_{IH} ( t ) \\ λ , & if {\hat{i}}_{f} ( t ; z ) < {X}_{IH} ( t )\end{matrix} \qquad [ Equation 3 ]$$

In other words, the extraction unit 130 compares the number of hidden infectious states (î_{f}(t;z)) according to the forward reasoning and the number of daily confirmed persons (X_{IH}).

In addition, if the number of hidden infectious states according to the forward reasoning (î_{f}(t;z)) is equal to or greater than the number of daily confirmed persons (X_{IH}), the extraction unit 130 calculates the loss function by subtracting the number of hidden infectious states (î_{f}(t;z))according to the forward reasoning from the number of hidden infectious states (î_{b}(t)) according to the backward reasoning, and then multiplying it to a power.

On the other hand, if the number of hidden infectious states (î_{f}(t;z)) according to the forward reasoning is less than the number of daily confirmed persons (X_{IH}), the extraction unit 130 uses the penalty term (λ) as the loss function. In this case, the penalty term (λ) indicates that the state value does not become negative.

Then, the extraction unit 130 calculates the inverse scale coefficient (z) by substituting the loss function ( [symbol] (t;z)) in Equation 4 below.

$${z}^{*} = arg {min}_{z} ( \frac{1}{T + 1} {\sum }_{t = 2}^{T + 2} ℒ ( t ; z ) ) \qquad [ Equation 4 ]$$

For example, assuming that the loss function (L) is calculated using data from May 1 to May 5, each of the loss function (L) of May 3, the loss function of May 4, and the loss function (L) of May 5 is calculated, and then an average of the loss functions Ls is calculated. In addition, the extraction unit 130 searches for the inverse scale coefficient z having the smallest loss function (argmin) L by repeating the process of calculating the loss function (L).

When step S230 is completed, the extraction unit 130 calculates a reproduction factor using the extracted inverse scale coefficient (S240).

The extraction unit 130 calculates the reproduction factor (U(t;z)) using Equation 5 below.

$$𝒰 ( t ; z ) = \frac{1}{z} \frac{{X}_{IH} ( t )}{{\sum }_{i = 1}^{i} ( {X}_{IH} ( t - l ) w ( l ) )} \frac{s ( t ) - {X}_{SV} ( t )}{{N}^{*} ( t )} , \qquad [ Equation 5 ]$$

Here, s indicates the number of susceptible persons, X_{SV} indicates the number of daily vaccine inoculated person, and N*(t) indicates the total population at the time point t.

Next, the prediction unit 150 predicts the number of hidden infectious persons at the time point t+1 using the reproduction factor (U(t;z)) calculated in step S240 (S250).

In other words, the prediction unit 150 calculates all state values by substituting the calculated reproduction factor (U(t;z)) into Equation 6 below.

$$[ \begin{matrix}υ ( t + 1 ) \\ s ( t + 1 ) \\ e ( t + 1 ) \\ i ( t + 1 ) \\ h ( t + 1 ) \\ r ( t + 1 ) \\ d ( t + 1 )\end{matrix} ] = [ \begin{matrix}1 - ξ & θ ( t ) & 0 & 0 & 0 & 0 & 0 \\ 0 & 1 - θ ( t ) & 0 & - 𝒰 ( t ; z ) & 0 & 0 & 0 \\ 0 & 0 & 1 - ϵ & 𝒰 ( t ; z ) & 0 & 0 & 0 \\ 0 & 0 & ϵ & ( 1 - φ ( t ) ) ( 1 - γ ) & 0 & 0 & 0 \\ 0 & 0 & 0 & φ ( t ) & ( 1 - δ ( t ) ) ( 1 - ρ ( t ) ) & 0 & 0 \\ ξ & 0 & 0 & ( 1 - φ ( t ) ) γ & ρ ( t ) ( 1 - δ ( t ) ) & 1 & 0 \\ 0 & 0 & 0 & 0 & δ ( t ) & 0 & 1\end{matrix} ] [ \begin{matrix}υ ( t ) \\ s ( t ) \\ e ( t ) \\ i ( t ) \\ h ( t ) \\ r ( t ) \\ d ( t )\end{matrix} ] \qquad [ Equation 6 ]$$

where

$$\frac{1}{ξ}$$

is the average duration time of immunity in the body,

$$\frac{1}{ϵ}$$

is the average latent period,

$$\frac{1}{γ}$$

is the average infection duration, δ(t) is the mortality rate, ρ(t) is the recovery rate of hospitalized patients, φ(t) indicates the detected case ratio, and θ(t) indicates the protective inoculation ratio of the first inoculation.

Then, the prediction unit 150 predicts the number of hidden infectious states at the time point t+1 using all the calculated state values. Also, the prediction unit 150 updates and stores all the calculated state values.

Meanwhile, the infectious disease transmission estimation system 100 according to the embodiment of the present disclosure may use the reproduction factor (U(t;z)) calculated in step S240 to evaluate the risk of the infectious disease.

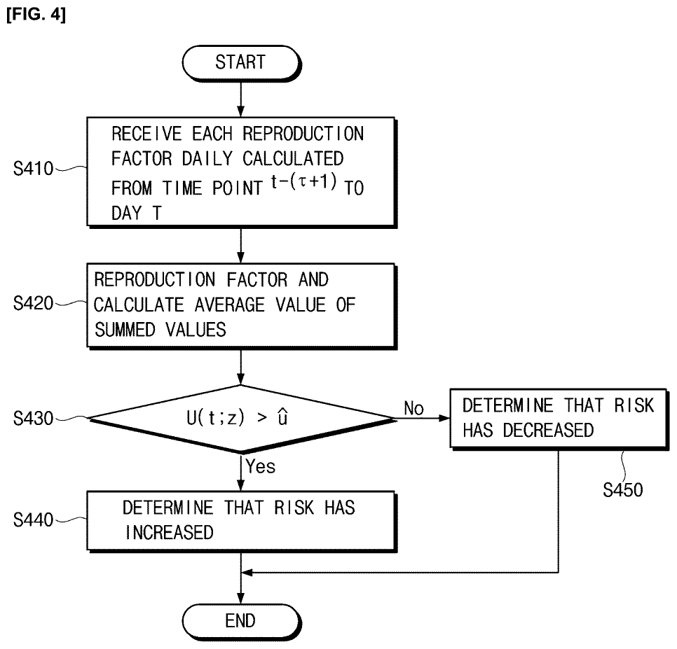

FIG. 4 is a flowchart illustrating a method for evaluating the risk of the infectious disease using the infectious disease transmission estimation system according to the embodiment of the present disclosure.

As illustrated in FIG. 4, the infectious disease transmission estimation system 100 according to the embodiment of the present disclosure receives a plurality of reproduction factors (U(t;z)) calculated until the time point t (S410).

For example, it is assumed that the risk of the infectious disease for τ days is measured. Then, the risk estimation unit 140 receives each reproduction factor ((U(t−(τ+1):t;z))) calculated daily from the time point t−(τ+1) to the t day.

Then, the risk estimation unit 140 calculates the average value of each of the input reproduction factors ((U(t−(τ+1):t−1; z))) (S420).

In other words, the reproduction factors ((U(t−(τ+1):t−1;z))) are summed from the time point ((t−(τ+1)) to the t−1 day, and the average value (û) of the summed values is calculated.

For example, it is assumed that τ is 3 days from May 3 to May 5. Then, the risk estimation unit 140 calculates the average value (û) by adding up a plurality of reproduction factors (U(t−(τ+1):t−1; z)) acquired during May 3 and May 4, and then dividing by 2.

When step S420 is completed, the risk estimation unit 140 compares the calculated average value (û) with the reproduction factor (U(t;z)) at the time point t (S430).

For example, the reproduction factor acquired on May 5 and the calculated average value (û) are mutually compared.

As a result of the comparison, if the reproduction factor (U(t;z)) at the time point t is greater than the average value (û), the risk estimation unit 140 determines that the risk for the infectious disease has increased (S440).

On the other hand, if the reproduction factor (U(t;z)) at the time point t is smaller than the average value (û) the risk estimation unit 140 determines that the risk for the infectious disease has decreased (S450).

The risk estimation unit 140 outputs the determination result to a monitoring device (not illustrated), and completes the evaluation of the infectious disease risk.

As described above, according to the present disclosure, it is possible to estimate the number of hidden infectious persons that cannot be officially counted, such as the number of infected persons in the latent state and the number of susceptible people by expanding the existing compartment model to 7 states in accordance with the quarantine policy for infectious disease. In addition, it is possible to quantitatively analyze the daily changing pattern of the infectious disease transmission using time-variable parameters.

In addition, according to the present disclosure, since the number of populations corresponding to each state may be estimated, it is possible to quantitatively explain the epidemic pattern of the infectious disease. Therefore, the present disclosure has great flexibility in that it is effective in allocating medical resources and manpower, applicable to newly discovered infectious diseases, and capable of being modified in consideration of alternative policies for infectious diseases.

Although the present disclosure has been described with reference to the embodiment illustrated in the drawings, this is merely exemplary, and it will be understood by those skilled in the art that various modifications and equivalent other embodiments are possible therefrom. Therefore, the true technical protection scope of the present disclosure should be determined by the technical spirit of the following claims.

What is claimed is:

1. A system for estimating for infectious disease transmission comprising:
a parameter receiving unit that constructs a discrete-time Markov chain model indicating a state and a state transition probability, and receives a parameter indicating status information according to the infectious disease transmission at a time point after t days have elapsed from a start of infection spread;
a calculation unit that calculates the number of hidden infectious states through backward reasoning, and calculates the number of hidden infectious states through forward reasoning using the received parameter;
an extraction unit that extracts an inverse scale coefficient using the calculated number of infection states and calculates a reproduction factor using the extracted inverse scale coefficient; and
a prediction unit that updates infectious disease status information using the calculated reproduction factor, and predicts the number of hidden infectious persons using the updated infectious disease status information.

2. The system for estimating for infectious disease transmission according to claim 1,
wherein the parameter includes at least one of the number of daily confirmed persons, the number of daily primary vaccine inoculated persons, the number of daily hospitalized and recovered persons, the number of hospitalized persons, the number of deaths, the number of susceptible persons corresponding to day 0, and the number of exposed persons, the number of recovered persons, the number of vaccinated states, and the number of hidden infectious states.

3. The system for estimating for infectious disease transmission according to claim 2,
wherein the calculation unit calculates the number of hidden infectious states (î_{b}through the backward reasoning using Equation below:
$${\hat{i}}_{b} ( t ) = \frac{1}{{φ}^{*}} {X}_{IH} ( t ) + {\sum }_{q = 1}^{t - 1} {( ( \frac{1}{{φ}^{*}} - 1 ) ( 1 - γ ) )}^{q + 1} {X}_{IH} ( t - q )$$
here, φ* indicates an arbitrarily assumed detected case ratio and X_{IH} indicates the number of daily confirmed persons.

4. The system for estimating for infectious disease transmission according to claim 2,
wherein the calculation unit calculates the number of hidden infectious states (î_{f}) through the forward reasoning using Equation below:
î_{f}(t; z)=ϵ(1−ϵ)e(t−2)+ϵU(t−2; z)i(t−2)+(1− [symbol] )i(t−1)−(1− [symbol] )X_{IH}(t−1)
here,
$$\frac{1}{γ}$$
indicates the average infection period, z indicates the inverse scale coefficient,
$$\frac{1}{ϵ}$$
indicates the average latent period, e indicates the exposed person, and i indicates the hidden infectious person.

5. The system for estimating for infectious disease transmission according to claim 2,
wherein the extraction unit calculates a loss function by substituting the number of hidden infectious states (î_{b}) calculated through the backward reasoning and the number of hidden infectious states (î_{f})calculated through the forward reasoning into Equation below:
$$ℒ ( t ; z ) = \{ \begin{matrix}{{\hat{i}}_{b} ( t ) - {\hat{i}}_{f} ( t ; z ) )}^{2} , & if {\hat{i}}_{f} ( t ; z ) \geq {X}_{IH} ( t ) \\ λ , & if {\hat{i}}_{f} ( t ; z ) < {X}_{IH} ( t )\end{matrix}$$

6. The system for estimating for infectious disease transmission according to claim 5,
wherein the extraction unit extracts the inverse scale coefficient (z) by substituting the calculated loss function into Equation below:
$${z}^{*} = arg {min}_{z} ( \frac{1}{T + 1} {\sum }_{t = 2}^{T + 2} ℒ ( t ; z ) )$$

7. The system for estimating for infectious disease transmission according to claim 6,
wherein the extraction unit calculates the reproduction factor (U(t;z)) using Equation below:
$$𝒰 ( t ; z ) = \frac{1}{z} \frac{{X}_{IH} ( t )}{{\sum }_{i = 1}^{i} ( {X}_{IH} ( t - l ) w ( l ) )} \frac{s ( t ) - {X}_{SV} ( t )}{{N}^{*} ( t )} ,$$
here, s indicates the number of susceptible persons, X_{SV} indicates the number of daily vaccine inoculated persons, and N*(t) indicates the total population at the time point t.

8. The system for estimating for infectious disease transmission according to claim 7,
wherein the risk estimation unit acquires each of reproduction factors calculated during a period τ and calculates the average value of the reproduction factors, and
determines that the risk for the infectious disease has increased when the calculated average value is greater than the reproduction factor calculated at the time point t.

9. The system for estimating for infectious disease transmission according to claim 7,
wherein the prediction unit uses the reproduction factor to update each state data for the vaccinated state, the susceptible state, the exposed state, the hidden infectious state, the hospitalized state, the recovery state, and the dead state, and
predicts the number of hidden infectious persons at a time point t+1 using the updated state data.
